(19) United States
(12) Patent Application Publication
(10) Pub. No.: US 2012/0122976 A1
(43) Pub. Date: May 17, 2012

(54) PHOSPHATE-FREE PHARMACEUTICAL COMPOSITION FOR THE TREATMENT OF GLAUCOMA

(75) Inventor: Frank Holzer, St. Ingbert (DE)

(73) Assignee: URSAPHARM ARZNEIMITTEL GMBH, Saarbrucken (DE)

(21) Appl. No.: 13/320,459

(22) PCT Filed: May 14, 2010
(86) PCT No.: PCT/EP10/02981

§ 371 Date: Feb. 2, 2012

(30) Foreign Application Priority Data
May 14, 2009 (DE) 10 2009 021 372.4

(51) Int. Cl. A61K 31/5575; A61P 27/06; A61K 31/165; A61K 31/216; A61K 31/16

(52) U.S. Cl. 514/530; 514/573; 514/613; 514/622

(57) ABSTRACT

The invention relates to a phosphate-free pharmaceutical composition which comprises at least one FP prostanoid receptor agonist and/or at least one prostamide receptor agonist and also citrate salts and/or citric acid.

The invention relates to a phosphate-free pharmaceutical composition which comprises at least one FP prostanoid receptor agonist and/or at least one prostamide receptor agonist and also citrate salts and/or citric acid.

Glaucomas which are also termed green star can lead to a loss of retinal ganglion cells and optic nerve fibres up to complete blinding because of increased intraocular pressure. The pressure building up intraocularly can be attributed to an impairment in the outflow of aqueous humour from the anterior chamber and the posterior chamber of the eye. Normally, the aqueous humour secreted by the ciliary body epithelium leaves the eye via the uveoscleral and the trabecular outflow.

There are used for the therapy of glaucoma, for example parasympathomimetic agents, such as for example pilocarpine, or sympathomimetic agents, such as for example dipivefrin.

Betablockers are also used to reduce the intraocular pressure, such as timolol, and carbonic anhydrase inhibitors, such as dorzolamide, which effect throttling of the inflow or a reduction in the production of the aqueous humour in the chamber.

Also prostaglandin analogues, such as latanoprost, tafluprost and travoprost, and also the prostamide bimatoprost have been used for several years in the therapy of glaucomas.

The therapy of glaucoma generally involves a long-term treatment.

The use of at least one calcium chelating agent and at least one ophthalmological viscosity regulator for the production of a phosphate-free pharmaceutical composition for the treatment and/or prevention of epithelial defects in the cornea and/or conjunctiva of the eye is known from PCT/EP2006/011053.

It has now emerged that, with long-term treatments of glaucoma patients by topical application of conventional eye drops or eye sprays on the eye surface, the result can be an impairment in visual capacity due to clouding of the cornea. This clouding of the cornea can be caused by inclusions and/or deposits of poorly soluble calcium phosphates which are included or deposited in or on the cornea and also the conjunctiva of the eye. This degeneration of the cornea of the eye is also termed corneal band degeneration or band keratopathy. Even slight inclusions and/or deposits of poorly soluble calcium phosphates in or on the cornea of the eye lead to massively increased sensitivity to bright light which can be attributed to light scattering effected on the inclusions or deposits of calcium phosphate(s) or of poorly soluble calcium compounds. In particular night vision is greatly impaired as a result.

There is therefore a requirement for a pharmaceutical composition which enables long-term treatment of glaucoma without the previously mentioned side-effects.

The object underlying the invention is achieved by providing a phosphate-free pharmaceutical composition, the phosphate-free pharmaceutical composition comprising at least one FP prostanoid receptor agonist and/or at least one prostamide receptor agonist and also citrate salts and/or citric acid.

Preferred developments are indicated in the sub-claims 2 to 18.

The object underlying the invention is achieved furthermore by the use of the phosphate-free composition according to the invention for the production of a medicine for the therapy and/or prevention of glaucoma.

There are understood by an FP prostanoid receptor, receptors to which prostaglandin F₂ₐₗₚₕₐ analogues and/or prostaglandin F₂ₐₗₚₕₐ binds.

There are understood according to the invention by the term FP prostanoid receptor, also FP receptors to which prostaglandin F₂ₐₗₚₕₐ bind. The FP receptors concern G-protein-coupled receptors, the endogenous physiological activator of which is prostaglandin F₂ₐₗₚₕₐ. In the case of stimulation of the FP prostanoid receptor, the result is an increase in the outflow of aqueous humour from the chamber, in particular via the uveoscleral route.

In addition to stimulation of the FP prostanoid receptor, an outflow of aqueous humour from the interior of the eye can also be effected by stimulation of the prostamide receptor. The prostamides concern prostaglandin F₂ₐₗₚₕₐ-1-amides. Without wishing to be bound to one theory, it is assumed that prostamide binds to the prostamide receptor and/or the prostanoid receptor and leads to outflow of the aqueous humour from the interior of the eye, in particular via the uveoscleral route.

The inventors have now established surprisingly that topical application of at least one FP prostanoid receptor agonist and/or at least one prostamide receptor agonist in combination with citrate salts and/or citric acid enables a completely surprising long-term treatment of the eye without the undesired side-effects of calcification or furring up of the cornea and/or the conjunctiva of the eye thereby occurring.

Citrates, i.e. salts of citric acid, and citric acid act inter alia as calcium chelating agent. Since the pharmaceutical composition according to the invention is phosphate-free, no additional phosphate is applied to the surface of the eye, on the one hand. On the other hand, calcium ions, in particular Ca²⁺ ions, are complexed by the citric acid or the citrates so that formation of poorly soluble calcium phosphates and/or calcium compounds is counteracted or their formation is preferably prevented.

Conventionally, eye drops or eye solutions are preferably phosphate-buffered since the phosphate buffer is a very stable buffer system with very good buffer capacity which is in particular very stable in storage.

Citric acid or citric salts, after solution in an aqueous medium, preferably in water, likewise form a stable buffer system with adequate buffer capacity although the buffer capacity is weaker in comparison with a phosphate buffer. The pH value of the pharmaceutical composition according to the invention is preferably in a physiological range, preferably in a range of pH 5.5 to 8.5, preferably 5.8 to 7.8, further preferred of 6.0 to 7.2. The pH value is possibly adjusted by the addition of acid or alkaline solution, preferably 0.1 N HCl or 0.1 N NaOH.

Both citric acid, primary, secondary and/or tertiary citrates can be used for the production of the citrate buffer. There are used as citrates, preferably alkali metal citrates, further preferred sodium citrates. Preferably, citric acid, sodium citrate, disodium citrate and/or trisodium citrate are used. The citrate buffer is present preferably in a concentration of 5 mmol/l to 100 mmol/l, further preferred of 10 mmol/l to 50 mmol/l.

According to an extremely preferred embodiment, the phosphate-free pharmaceutical composition is also calcium ion-free. Calcium ion-free in the sense of the invention means that the phosphate-free pharmaceutical composition comprises less than 0.3 mmol/l calcium ions, preferably less than 0.1 mmol/l calcium ions and in particular preferably no calcium ions.

There is understood by phosphate-free, in the sense of the invention, that the pharmaceutical composition comprises less than 7 mmol/l phosphate ions, preferably less than 3 mmol/l phosphate ions, particularly preferred less than 1 mmol/l phosphate ions and most particularly preferred no phosphate ions.

There are understood by the term “phosphate ions”, in the sense of the invention, in particular PO₄³⁻, HPO₄²⁻ and/or H₂PO₄⁻.

The phosphate-free pharmaceutical composition according to the invention, because of the absence of phosphate ions and because of the presence of citrate salts and/or citric acid, prevents the formation of calcium-phosphate complexes and/or calcium-phosphate compounds and/or other poorly soluble calcium compounds in the eye, which can lead to deposition or inclusion on or in the cornea and/or the conjunctiva of the eye and consequently to a significant restriction in visual capacity due to light scattering on the calcium-phosphate complexes and/or calcium-phosphate compounds and/or other poorly soluble calcium compounds. There are understood by poorly soluble calcium compounds, in particular compounds which form deposits and/or inclusions in the cornea.

The citrate salts and/or citric acid complex calcium ions also physiologically present in the eye and hence counteract the production of poorly soluble calcium compounds which can lead to deposits or inclusions in the cornea and/or conjunctiva of the eye.

The phosphate-free pharmaceutical composition according to the invention prevents or reduces calcification in the cornea and/or in the conjunctiva of the eye.

Furthermore, the citrate salts and/or citric acid or the citrate buffer produced therefrom surprisingly also have the effect of promoting wound-healing. Hence, the phosphate-free pharmaceutical composition according to the invention assists regeneration of any defects of the eye surface which are caused for example by preservatives and/or an inadequate tear film, in particular the corneal surface and/or conjunctiva of the eye.

It has been shown that citric acid or the citrates, because of their complexing properties, in particular due to the complexing of calcium ions, effect improved penetration of the FP prostanoids or prostamides from the epithelial side in and through the cornea. It is suspected that because of the complexing of calcium ions, the result is changes in the intracellular matrix material of the cornea. It is suspected that these changes effect loosening of the tight junctions which are also termed Zonula occludens. The loosening of the tight junctions makes it possible then that the relatively hydrophobic FP prostanoids or prostamides can reach the eye interior more easily in and through the cornea.

The use of citric acid or citrates in combination with FP prostanoid(s) and/or prostamide(s) leads therefore to a surprising synergistic effect. On the one hand, citric acid and/or citrates counteract a previously damaged cornea, for example because of the syndrome of dry eye, frequently occurring in elderly patients, and/or by using preservative-containing eye drops, due to the properties which promote wound-healing. On the other hand, the citric acid and/or the citrates, because of loosening the tight junctions due to the complexing of calcium ions, effect improved absorption of the FP prostanoids or prostamides into the eye. As a result, the dwell time of the FP prostanoids or prostamides on the cornea which is required for absorption of the FP prostanoids and/or prostamides is reduced on the one hand. As a result, in particular also the possibility of washing out of the FP prostanoids and/or prostamides from the eye surface, caused by the natural tear flow of the eye, is reduced and hence the bioavailability of the FP prostanoids or prostamides in the interior of the eye is increased. The improved absorption of the FP prostanoids or prostamides makes it possible to reduce the concentration of the quantity of FP prostanoids or prostamides to be applied to the cornea.

The above-mentioned FP prostanoids concern in particular latanoprost, travoprost and/or tafluprost, and the above-mentioned prostamides concern in particular bimatoprost.

This synergistic effect of citric acid or citrates in conjunction with FP prostanoids or prostamides is unexpected. As a result, irritation to the eye surface is reduced or avoided.

The invention also relates to the use of FP prostanoids, in particular latanoprost, travoprost and/or tafluprost, and/or prostamide, in particular bimatoprost, in combination with citric acid and/or citrate for the provision of an increased concentration of the mentioned active substance/substances in the aqueous humour of the eye, in the iris and/or in the ciliary body.

It has therefore emerged surprisingly that the phosphate-free pharmaceutical composition for the eye, according to the invention, is tolerated very well, in particular causes no substantial irritation to the eye or to the eye surface.

The FP prostanoid receptor agonist and/or the prostamide receptor agonist are preferably used in a concentration in a range of 0.00001% by weight to 0.05% by weight, further preferred of 0.00005% by weight to 0.01% by weight, even further preferred of 0.0001% by weight to 0.005% by weight, respectively relative to the total weight of the composition.

The phosphate-free pharmaceutical composition according to the invention enables a reduction in the intraocular pressure in a range of 20 to 40%, normally in a range of 20 to 30%, relative to the intraocular pressure before application.

The frequency of application of the phosphate-free pharmaceutical composition according to the invention is effected as a function of the individual requirement or the severity of the glaucoma. Thus in the case of eye drops, 1 to 3 drops per eye, preferably once daily, can be sufficient. In the case of severe glaucoma disease, also more drops, for example up to 16 drops, also several times daily, can be applied per eye.

According to a further preferred embodiment, the at least one FP prostanoid receptor agonist and/or the at least one prostamide receptor agonist concern compounds with the structural formula (I) or (II):

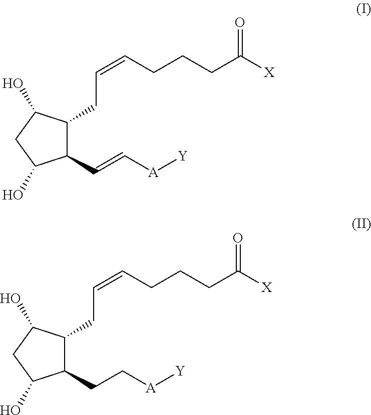

wherein

X stands for, independently of each other, OR₃ or NR₁R₂, wherein R₁, R₂, R₃ stand for, independently of each other, H, unbranched or branched alkyl radical with 1 to 8 carbon atoms, preferably 2 to 6 carbon atoms, unbranched or branched OH/substituted alkyl radical with 1 to 8 carbon atoms, preferably 2 to 6 carbon atoms;

A stands for, independently of each other, CHOH, C═O or CF₂;

Y stands for, independently of each other,

unbranched or branched alkyl radical with 1 to 10 carbon atoms, preferably 3 to 8 carbon atoms,
or for unbranched or branched alkylaryl radical with 6 to 12 carbon atoms, preferably with 7 to 10 carbon atoms, aryl preferably being phenyl,
or for trifluoromethyl-substituted alkylaryl radical with 7 to 12 carbon atoms, preferably with 8 to 9 carbon atoms, aryl preferably being phenyl,
or for

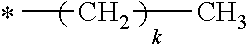

wherein k stands for a whole number from 0 to 9, preferably 2 to 7,
or for

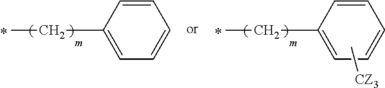

wherein m stands for a whole number from 0 to 6, preferably 2 to 4 and wherein Z stands for H or F,

or for

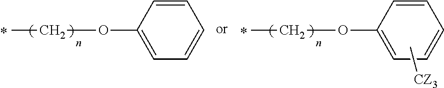

wherein n stands for a whole number from 0 to 4, preferably 1 to 3 and wherein Z stands for H or F.

The above-indicated FP prostanoid receptor agonists or prostamide receptor agonists can also be present as pharmaceutical acceptable salts and/or esters.

According to a preferred embodiment of the invention, the FP prostanoid receptor agonist is selected from the group which consists of prostaglandin, prostaglandin analogue and mixtures thereof.

According to a preferred development of the invention, the prostaglandin concerns prostaglandin F₂ₐₗₚₕₐ with the structural formula (III). The prostaglandin F₂ₐₗₚₕₐ is also termed dinoprost:

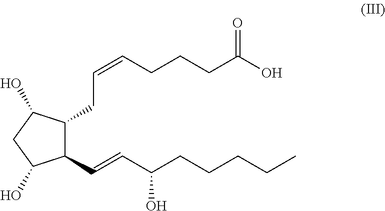

Prostaglandin F₂ₐₗₚₕₐ or dinoprost can also be present as pharmaceutically acceptable ester, for example as alkyl ester with an alkyl radical of 1 to 6 carbon atoms. Preferably, the alky ester concerns an ethyl ester or isopropyl ester. The isopropyl ester has proved to be very suitable.

According to a further preferred embodiment, the prostaglandin analogue concerns a prostaglandin F₂ₐₗₚₕₐ analogue.

According to a further preferred embodiment, the prostaglandin F₂ₐₗₚₕₐ analogue is selected from the group which consists of latanoprost (structural formula (IV)), tafluprost (structural formula (V)), travoprost (structural formula (VI)), unoprostone (structural formula (VII)), and mixtures and also their pharmaceutically acceptable salts and esters thereof.

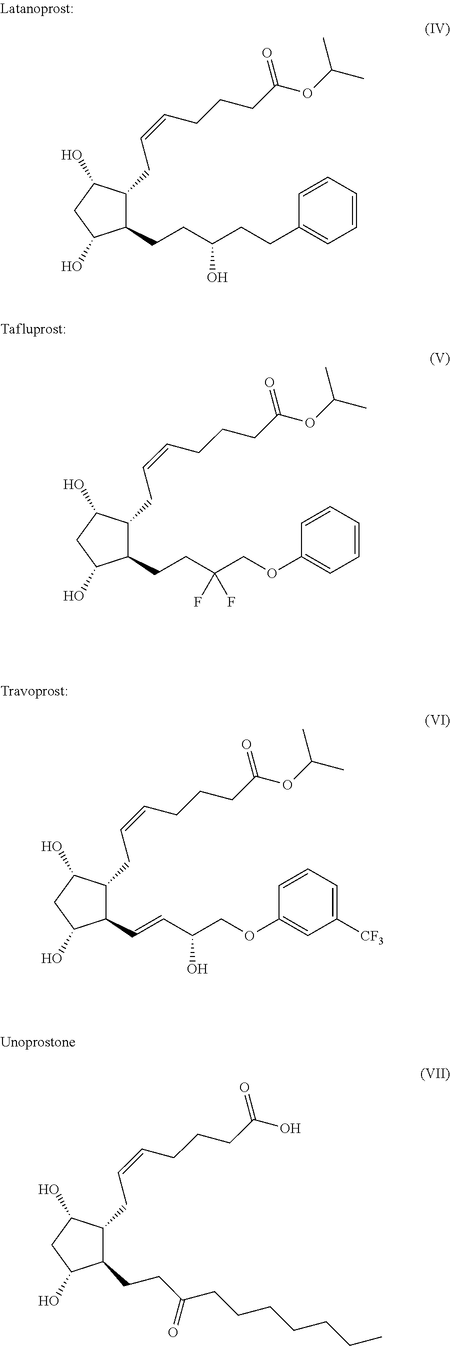

Unoprostone can also be present as alkyl ester. According to a preferred embodiment, this hereby concerns an ethyl- or isopropyl ester.

According to a further variant of the invention, the prostaglandin F₂ₐₗₚₕₐ analogue is a 15-keto-prostaglandin F₂ₐₗₚₕₐ analogue and is preferably selected from the group which consists of 15-keto-latanoprost, 15-keto-travoprost and mixtures and also their pharmaceutically acceptable salts and esters thereof. In this variant, the OH group on the C₁₅ in the prostaglandin F₂ₐₗₚₕₐ analogue is replaced by a keto group.

Prostaglandin F₂ₐₗₚₕₐ can be present, according to a variant of the invention, as amide or else as a pharmaceutically acceptable salt thereof. The pharmaceutically acceptable salt can for example be a chloride, acetate, sulphate or mixed salts thereof etc. The prostaglandin F₂ₐₗₚₕₐ amide is also termed prostamide.

According to a preferred embodiment, the prostamide receptor agonist is prostamide or a prostamide analogue.

Preferably, the prostamide is prostaglandin F₂ₐₗₚₕₐ amide and has the following structural formula (VIII):

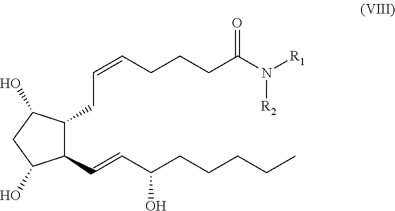

wherein R₁ and R₂ stand for, independently of each other, hydrogen, alkyl or hydroxyalkyl with 1 to 8 carbon atoms, preferably 2 to 4 carbon atoms.

The substituents R₁ and R₂ are preferably, independently of each other, hydrogen, methyl, ethyl, n-propyl, isopropyl, n-butyl, sec.-butyl, n-pentyl, n-hexyl, n-heptyl or n-octyl. According to a further preferred embodiment, the above-mentioned preferred alkyl groups are substituted with at least one OH group. Preferably the OH group is disposed terminally, i.e. at the end of the alkyl group situated far away from the nitrogen.

According to a further preferred embodiment, R₂ is hydrogen and R₁ is alkyl or hydroxyalkyl with 1 to 8 carbon atoms, preferably with 2 to 4 carbon atoms. R₁ is thereby preferably methyl, ethyl, n-propyl, isopropyl, n-butyl, sec.-butyl, n-pentyl, n-hexyl, n-heptyl or n-octyl. According to a further preferred embodiment, the above-mentioned preferred alkyl groups are substituted with at least one OH group. Preferably, the OH group is disposed terminally, i.e. at the end of the alkyl group R₁ situated far away from the nitrogen.

The substituted amide can thereby be present respectively also as a pharmaceutically acceptable salt or acceptable ester thereof. The pharmaceutically acceptable salt can be for example a chloride, acetate, sulphate, or mixed salts thereof etc.

According to a further preferred embodiment, the prostaglandin F₂ₐₗₚₕₐ amide concerns bimatoprost (structural formula (IX)) or prostaglandin F₂ₐₗₚₕₐ-1-ethanol amide (structural formula X)) or the pharmaceutically acceptable salts or esters thereof.

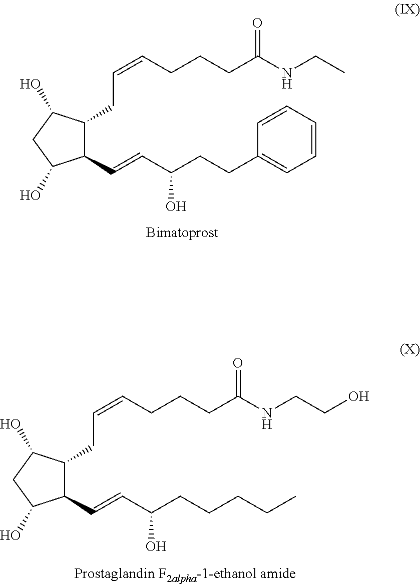

According to a further variant of the invention, the prostaglandin F₂ₐₗₚₕₐ amide analogue is a 15-keto-prostaglandin F₂ₐₗₚₕₐ amide analogue and is selected preferably from the group which consists of 15-keto-bimatoprost, 15-keto-prostaglandin F₂ₐₗₚₕₐ-1-ethanol amide and mixtures and also their pharmaceutically acceptable salts and esters thereof. In this variant, the OH group on the C₁₅ in the prostaglandin F₂ₐₗₚₕₐ amide analogue is replaced by a keto group.

According to a preferred development, the phosphate-free pharmaceutical composition according to the invention comprises at least one ophthalmologically tolerable viscosity regulator.

There are termed viscosity regulators in the sense of the invention, substances which have a viscosity-increasing effect.

In the sense of the invention, there is understood by “ophthalmologically tolerable”, in particular that no irritations to the eye and possibly no impairments in visual capacity occur.

Preferably, the viscosity regulator has a viscoeleastic behaviour. There is understood by a viscoelastic behaviour according to the invention that the viscosity changes under the influence of compressive, tensile, thrust and/or shear stresses. For particular preference, the phosphate-free pharmaceutical composition according to the invention has the behaviour of a non-Newtonian liquid as a result of the viscosity regulator.

The viscosity is preferably in a range of 2 to 1,000 mPa·s, further preferred in a range of 2 to 500 mPa·s, particularly preferred in a range to 2 to 100 mPa·s.

The viscosity-increasing effect has the extremely advantageous effect that the phosphate-free pharmaceutical composition applied to the eye surface has an increased dwell time and flows off the eye surface again more slowly. The non-Newtonian flow behaviour of the viscosity regulator produces a property which is excellent for application to the eye, namely that the viscosity reduces with increasing shearing rate. After application of the phosphate-free pharmaceutical composition with the viscosity regulator on the surface of the eye, a shear stress is applied to the phosphate-free pharmaceutical composition via blinking of the eyelid, as a result of which the initially increased viscosity is reduced. Due to blinking of the eyelid, the viscosity is reduced so that a uniform film forms on the surface of the eye. After blinking, the viscosity increases so that the film on the eye surface adheres well and runs off only slowly, as a result of which the dwell time of the phosphate-free composition according to the invention on the eye surface is increased.

Because of the increased dwell time on the eye surface, the bioavailability of the at least one FP prostanoid receptor agonist and/or of the at least one prostamide receptor agonist can be increased since rapid outflow of the at least one FP prostanoid receptor agonist and/or of the at least one prostamide receptor agonist is prevented and therefore the period of time available for absorption of the active substances is lengthened.

Preferably, the viscosity regulator acts at the same time as sliding aid and lubricant on the eye. The sliding and lubricating effect is advantageous in particular when the eye surface, in particular the cornea, has already suffered damage, in particular epithelium lesions. Hence the use of a viscosity regulator is advantageous in particular when an epithelium lesion has already been effected because of long-term therapy with conventional pharmaceutical compositions.

According to a preferred embodiment, the quantity of viscosity regulator is approx. 0.005% by weight up to approx. 5% by weight, preferably approx. 0.01% by weight up to approx. 1% by weight, respectively relative to the total weight of the phosphate-free pharmaceutical composition.

According to a preferred development of the invention, the ophthalmologically tolerable viscosity regulator is selected from the group which consists of chondroitin sulphate, polyacrylamide, polyacrylic acid, polyacrylic resins, polyethylene glycol, cellulose derivatives, polysaccharides, polyvinyl pyrrolidone, hyaluronic acid, hyaluronates, derivatives thereof and mixtures thereof.

Hyaluronic acid and the salts thereof, the hyaluronates, have proved to be very suitable.

Hyaluronic acid is a component of the vitreous body of the eye and in this respect does not represent a foreign compound to the human organism. For this reason, hyaluronic acid is very readily tolerated from an immunological point of view. Furthermore, hyaluronic acid or hyaluronate has a structural similarity to mucin. Mucin forms the lowermost layer of the three-layer tear film and ensures optimum wetting of the corneal and conjunctival epithelia.

Furthermore, hyaluronic acid has an excellent property for application to the eye, namely that the viscosity decreases with an increase in the shearing rate. Hyaluronic acid hence has a non-Newtonian flow behaviour.

Hyaluronic acid or the salts thereof, hyaluronates or in particular sodium hyaluronate, has or have excellent optical properties so that no impairment in the visual capacity results in the patients who are treated.

Hyaluronic acid or hyaluronate can be isolated from the vitreous body of the eyes of cattle or else also from cockscombs. Furthermore, hyaluronic acid or hyaluronate can be produced also in strains of bacteria with pharmaceutical quality. For example potassium-, sodium- and/or magnesium hyaluronate can be used as salts of hyaluronic acid. Sodium hyaluronate is particularly preferred.

Aqueous sodium hyaluronate solutions and/or hyaluronic acid are exceptionally suitable, on the basis of these physical properties, as sliding aid and lubricant with a good adhesive effect and extended dwell time on the conjunctival and corneal epithelia without impairing visual performance.

According to a further embodiment, hyaluronic acid and/or hyaluronate has a molecular weight which is in a range of 50,000 to 10,000,000 dalton, preferably of approx. 250,000 to 5,000,000. The molecular weight of hyaluronic acid or hyaluronate is particularly preferably 50,000 to 4,000,000 dalton. For exceptional preference, the hyaluronic acid or hyaluronate has a molecular weight of approx. 1,500,000 to 3,500,000 dalton. Hyaluronic acid and/or hyaluronate are used preferably in a concentration of 0.01 to 1.0% by weight, further preferred of 0.05 to 0.8% by weight, particularly preferred of 0.08 to 0.4% by weight, respectively relative to the total weight of the phosphate-free pharmaceutical composition.

The high molecular weight of hyaluronic acid or of the hyaluronate used, such as for example sodium hyaluronate, effects high viscoelasticity at a low concentration. In the solution, the molecular chains are present in a random arrangement in the manner of a tangle. Under the influence of the shear forces exerted by movement of the eyelid, the macromolecules align themselves approximately parallel. This change in the three-dimensional structure under the influence of shear forces must be crucial for the excellent viscoelastic properties.

According to a further preferred embodiment, the pharmaceutical composition comprises phosphate-free pharmaceutical auxiliary substances which are selected from the group which consists of inorganic buffer substances, organic buffer substances, inorganic salts, organic salts, solvents, solubility aids, solubility promoters, salt formers, viscosity and consistency regulators, gelatinising agents, emulsifiers, solubilisers, wetting agents, expansion aids, antioxidants, preservatives, filling and carrier substances, osmolarity regulators and also mixtures thereof.

The inorganic buffer substances are selected preferably from the group consisting of boric acid, sodium hydroxide, sodium borate, sodium carbonate, hydrochloric acid, sodium hydrogen carbonate and mixtures thereof.

The organic buffer substances are selected preferably from the group which consists of acetic acid, sodium acetate, potassium hydrogen phthalate, succinic acid, maleic acid, trometamol and mixtures thereof.

The inorganic salts are selected preferably from the group which consists of common salt, potassium chloride, aluminium hydroxide, ammonium hydroxide, ammonium chloride, ammonium sulphate, calcium chloride and mixtures thereof.

The organic salts are selected preferably from the group which consists of salts of succinic acid, salts of maleic acid, salts of acetic acid and mixtures thereof.

The emulsifiers, solubilisers, wetting agents and expansion agents are selected preferably from the group which consists of poloxamer, phospholipids, lecithin, alkali soaps, for example sodium palmitate, alkali sulphates, for example sodium lauryl sulphate, macrogols, for example polyethylene glycols, macrogol stearates, polysorbates, macrogol glycerol monostearates, propylene glycols, glycerine, cyclodextrines and mixtures thereof.

The antioxidants are selected preferably from the group which consists of ascorbic acid, butyl hydroxytoluene, alpha-tocopherol and their salts and esters and mixtures thereof.

The osmolality regulators are selected preferably from the group which consists of sorbitol, glucose, glycerine, polyethylene glycol, fructose and mixtures thereof.

There can be used as solvent, for example water, monovalent alcohols, paraffins, triglycerides, oils or mixtures thereof.

According to a development, the phosphate-free composition according to the invention can also comprise solubilisers, detergents and/or emulsifiers in order further to improve the solubility of the at least one hydrophobic FP prostanoid receptor agonist and/or prostamide receptor agonist. In order to avoid undesired irritation to the eye, as little quantities as possible of solubilisers, detergents, and/or emulsifiers are added, according to a preferred variant.

Preferably, the pharmaceutical composition according to the invention is formulated to be free of preservatives. In order to avoid undesired irritation to the eye, no preservatives, in particular no benzalkonium chloride, are added to the pharmaceutical composition according to the invention, according to a preferred variant.

Furthermore, it is preferred according to the invention that the phosphate-free pharmaceutical composition is present in the form of a solution, of drops, a spray, a suspension, emulsion, a gel, an ointment, paste, a powder, powder, granulate or a tablet.

The phosphate-free pharmaceutical composition is preferably an ophthalmic agent, further preferred an ophthalmic agent for topical application.

In the provision of the phosphate-free pharmaceutical composition in the form of eye ointments or eye gels, this is prepared for example in Vaseline or paraffin with and without the addition of an emulsifier, such as for example cholesterin, wool wax, wool wax alcohols, cetyl alcohol etc.

According to a preferred embodiment, the phosphate-free pharmaceutical composition is present in the form of a, preferably aqueous, solution so that this can be applied for example in the form of eye drops or an eye spray on the surface of the eye.

In one embodiment, the osmolarity of the phosphate-free pharmaceutical composition according to the invention is at 100 to 900 mOsm/l.

The preferably aqueous solutions are thereby isotonic solutions according to a preferred embodiment, relative to tear fluid. In the case of isotonic solutions, the osmolarity is preferably at 200 to 350 mOsm/l, preferably at 300 mOsm/l. According to a further preferred embodiment, the phosphate-free pharmaceutical composition according to the invention is hypoosmolar. In this case, the osmolarity can be for example approx. 160-180 mOsm/l. A hypoosmolar solution is used in particular when an abnormally high osmolarity of a tear film must be compensated for in the case of a patient with dry eyes. As a function of the symptoms to be treated, a hypertonic solution can also be advantageous. The pharmaceutical composition can thereby also have a particularly high osmolarity of 700 to 900 mOsm/l.

For the isotonisation of the aqueous solution, preferably sodium chloride, boric acid, sorbitol, glycerine, etc. are used.

The phosphate-free composition according to the invention is also suitable for the production of a combination preparation, the FP prostanoid(s) and/or prostamide(s) being present together with one or more further active substances. Hence, the present invention also relates to a pharmaceutical composition in the form of a combination preparation in which, in addition to FP prostanoid and/or prostamide, at least one further active substance or at least two further active substances are present.

According to a preferred development of the invention, the pharmaceutical composition according to the invention can comprise, in addition to FP prostanoid(s) (FP prostanoid receptor agonist(s)) and/or prostamide(s) (prostamide receptor agonist(s)), also beta blockers, carbonic anhydrase inhibitors, sympathomimetic agents, parasympathomimetic agents or mixtures thereof.

The above-mentioned FP prostanoids concern in particular latanoprost, travoprost and/or tafluprost, and the above-mentioned prostamides concern in particular bimatoprost which can be present respectively with the previously mentioned active substances as active substance combination according to the invention.

In particular timolol, betaxolol, carteolol, levobunolol and also their salts and/or esters or mixtures thereof have proved to be very suitable as betablockers.

Timolol is used preferably in the form of timolol hydrogen maleate.

In particular dorzolamide, brinzolamide, acetazolamide and also their salts and/or esters or mixtures thereof have proved to be very suitable as carbonic anhydrase inhibitors.

Dorzolamide is used preferably in the form of the hydrochloride (dorzolamide HCl).

In particular brimonidine, apraclonidine (Iopidine), acetazolamide and also their salts and/or esters or mixtures thereof have proved to be very suitable as sympathomimetic agents.

Brimonidine is used preferably as tartrate salt (brimonidine[(R,R)-tartrate], e.g. Alphagan.

Pilocarpine and also its salts and/or esters or mixtures thereof have proved to be very suitable as a parasympathomimetic agent.

Pilocarpine is used preferably in the form of pilocarpine nitrate.

According to a preferred variant of the invention, the active substance combination comprises FP prostanoid and timolol or prostamide and timolol:

latanoprost and timolol

travoprost and timolol

tafluprost and timolol

bimatoprost and timolol

According to a preferred variant of the invention, the active substance combination comprises FP prostanoid and timolol or prostamide and dorzolamide:

latanoprost and dorzolamide

travoprost and dorzolamide

tafluprost and dorzolamide

bimatoprost and dorzolamide

The above active substance combinations can of course comprise auxiliary substances, such as for example inorganic buffer substances, organic buffer substances, inorganic salts, organic salts, emulsifiers, solubilisers, wetting agents, expansion agents, antioxidants, osmolality regulators, etc. or mixtures thereof. The phosphate-free pharmaceutical composition according to the invention can be used in the therapy and/or prevention of glaucoma.

In particular, the phosphate-free pharmaceutical composition according to the invention is suitable for the production of a medicine for the therapy and/or prevention of glaucoma.

The pharmaceutical composition according to the invention is preferably present in a storage and dosage system. Preferably, it thereby concerns a multidose container or multidose system. The COMOD® system of the company Ursapharm, Saarbrücken, Germany has proved to be very suitable for this purpose.

According to a preferred variant, the container in which the pharmaceutical composition according to the invention is stored consists up to at least 75% by weight of polyethylene, preferably approx. 78 to 94% by weight of polyethylene, respectively relative to the total weight of the container. The remainder up to 100% by weight is preferably an alkene or polyalkene which is different from polyethylene.

According to a further preferred variant, the container in which the pharmaceutical composition according to the invention is stored consists up to at least 75% by weight of polypropylene, preferably approx. 78 to 94% by weight of polypropylene, respectively relative to the total weight of the container. The remainder up to 100% by weight is preferably an alkene or polyalkene which is different from polypropylene.

According to a further preferred variant, the container consists of glass.

The invention is explained subsequently with reference to examples without being restricted thereto.

### EXAMPLES

### Example 1

| | | |
|---|---|---|
| bimatoprost | 0.3 | mg |
| polyvinyl pyrrolidone (Kollidon) | 20.0 | mg |
| citric acid | 0.05 | mg |
| sodium citrate × 2 H₂O | 8.5 | mg |
| sorbite | 33 | mg |
| water for injection purposes (f.I.) | ad 1.0 | ml |
| adjusted to pH 7.4 with 0.1N NaOH | | |

### Example 2

| | | |
|---|---|---|
| bimatoprost | 0.3 | mg |
| hyaluronic acid, Na salt (MW: 2-3 · 10⁶ Da) | 0.1 | mg |
| citric acid | 0.05 | mg |
| sodium citrate × 2 H₂O | 8.5 | mg |
| sorbite | 33 | mg |
| water f.I. | ad 1.0 | ml |
| adjusted to pH 7.4 with 0.1N NaOH | | |

### Example 3

| | | |
|---|---|---|
| travoprost | 0.04 | mg |
| hyaluronic acid, Na salt (MW: 2-3 · 10⁶ Da) | 0.1 | mg |
| sorbite | 30 | mg |
| sodium citrate × 2 H₂O | 15 | mg |
| citric acid | 0.65 | mg |
| water f.I. | ad 1.0 | ml |
| adjusted to pH 6.0 with 0.1N HCl | | |

### Example 4

| | | |
|---|---|---|
| latanoprost | 0.05 | mg |
| hyaluronic acid, Na salt (MW: 2-3 · 10⁶Da) | 0.1 | mg |
| sorbite | 30 | mg |
| sodium citrate × 2 H₂O | 15 | mg |
| citric acid | 0.65 | mg |
| water f.I. | ad 1.0 | ml |
| adjusted to pH 6.0 with 0.1N HCl | | |

### Example 5

| | | |
|---|---|---|
| latanoprost | 0.05 | mg |
| polyvinyl pyrrolidone (Kollidon) | 20.0 | mg |
| sodium citrate × 2 H₂O | 7.5 | mg |
| citric acid | 0.2 | mg |
| sodium chloride | 6.0 | mg |
| water f.I. | ad 1.0 | ml |
| adjusted to pH 6.0 with 0.1N HCl | | |

### Example 6

| | | |
|---|---|---|
| tafluprost | 0.015 | mg |
| polyvinyl pyrrolidone (Kollidon) | 20.0 | mg |
| sodium citrate × 2 H₂O | 7.5 | mg |
| citric acid | 0.2 | mg |
| sodium chloride | 6.0 | mg |
| water f.I. | ad 1.0 | ml |
| adjusted to pH 6.0 with 0.1N HCl | | |

### Example 7

| | | |
|---|---|---|
| tafluprost | 0.05 | mg |
| hyaluronic acid, Na salt (MW: 2-3 · 10⁶Da) | 1.0 | mg |
| sodium citrate × 2 H₂O | 7.5 | mg |
| citric acid | 0.2 | mg |
| sodium chloride | 6.0 | mg |
| water f.I. | ad 1.0 | ml |
| adjusted to pH 6.0 with 0.1N HCl | | |

### Example 8

| | | |
|---|---|---|
| latanoprost | 0.05 | mg |
| sodium chloride | 6.0 | mg |
| sodium citrate × 2 H₂O | 10.0 | mg |
| citric acid | 0.2 | mg |
| water f.I. | ad 1.0 | ml |
| adjusted to pH 6.0 with 0.1N HCl | | |

### Example 9

| | | |
|---|---|---|
| latanoprost | 0.05 | mg |
| sodium chloride | 6.0 | mg |
| sodium citrate × 2 H₂O | 10.0 | mg |
| citric acid | 0.2 | mg |
| water f.I. | ad 1.0 | ml |
| adjusted to pH 6.0 with 0.1N HCl | | |

### Example 10

| | | |
|---|---|---|
| latanoprost | 0.05 | mg |
| sodium chloride | 6.0 | mg |
| sodium citrate × 2 H₂O | 10.0 | mg |
| citric acid | 0.2 | mg |
| water f.I. | ad 1.0 | ml |
| adjusted to pH 6.0 with 0.1N HCl | | |

### Example 11

| | | |
|---|---|---|
| bimatoprost | 0.3 | mg |
| timolol | 2.5 | mg |
| hyaluronic acid, Na salt (MW: 2-3 · 10⁶Da) | 0.1 | mg |
| citric acid | 0.05 | mg |
| sodium citrate × 2 H₂O | 8.5 | mg |
| sorbite | 33 | mg |
| water f.I. | ad 1.0 | ml |
| adjusted to pH 7.4 with 0.1N NaOH | | |

### Example 12

| | | |
|---|---|---|
| bimatoprost | 0.3 | mg |
| dorzolamide | 10 | mg |
| hyaluronic acid, Na salt (MW: 2-3 · 10⁶Da) | 0.1 | mg |
| citric acid | 0.05 | mg |
| sodium citrate × 2 H₂O | 8.5 | mg |
| sorbite | 33 | mg |
| water f. I. | ad 1.0 | ml |
| adjusted to pH 7.4 with 0.1N NaOH | | |

### Example 13

| | | |
|---|---|---|
| travoprost | 0.04 | mg |
| timolol | 2.5 | mg |
| hyaluronic acid, Na salt (MW: 2-3 · 10⁶Da) | 0.1 | mg |
| sorbite | 30 | mg |
| sodium citrate × 2 H₂O | 15 | mg |
| citric acid | 0.65 | mg |
| water f.I. | ad 1.0 | ml |
| adjusted to pH 6.0 with 0.1N HCl | | |

### Example 14

| | | |
|---|---|---|
| travoprost | 0.04 | mg |
| dorzolamide | 10 | mg |
| hyaluronic acid, Na salt (MW: 2-3 · 10⁶Da) | 0.1 | mg |
| sorbite | 30 | mg |
| sodium citrate × 2 H₂O | 15 | mg |
| citric acid | 0.65 | mg |
| water f.I. | ad 1.0 | ml |
| adjusted to pH 6.0 with 0.1N HCl | | |

### Example 15

| | | |
|---|---|---|
| latanoprost | 0.05 | mg |
| timolol | 2.5 | mg |
| hyaluronic acid, Na salt (MW: 2-3 · 10⁶Da) | 0.1 | mg |
| sorbite | 30 | mg |
| sodium citrate × 2 H₂O | 15 | mg |
| citric acid | 0.65 | mg |
| water f.I. | ad 1.0 | ml |
| adjusted to pH 6.0 with 0.1N HCl | | |

### Example 16

| | | |
|---|---|---|
| latanoprost | 0.05 | mg |
| dorzolamide | 10 | mg |
| hyaluronic acid, Na salt (MW: 2-3 · 10⁶Da) | 0.1 | mg |
| sorbite | 30 | mg |
| sodium citrate × 2 H₂O | 15 | mg |
| citric acid | 0.65 | mg |
| water f.I. | ad 1.0 | ml |
| adjusted to pH 6.0 with 0.1N HCl | | |

### Example 17

| | | |
|---|---|---|
| tafluprost | 0.05 | mg |
| timolol | 2.5 | mg |
| hyaluronic acid, Na salt (MW: 2-3 · 10⁶Da) | 1.0 | mg |
| sodium citrate × 2 H₂O | 7.5 | mg |
| citric acid | 0.2 | mg |
| sodium chloride | 6.0 | mg |
| water f.I. | ad 1.0 | ml |
| adjusted to pH 6.0 with 0.1N HCl | | |

### Example 18

| | | |
|---|---|---|
| tafluprost | 0.05 | mg |
| dorzolamide | 10 | mg |
| hyaluronic acid, Na salt (MW: 2-3 · 10⁶Da) | 1.0 | mg |
| sodium citrate × 2 H₂O | 7.5 | mg |
| citric acid | 0.2 | mg |
| sodium chloride | 6.0 | mg |
| water f.I. | ad 1.0 | ml |
| adjusted to pH 6.0 with 0.1N HCl | | |

1. A phosphate-free pharmaceutical composition,
comprising at least one FP prostanoid receptor agonist and/or at least one prostamide receptor agonist and citrate salts and/or citric acid.

2. The phosphate-free pharmaceutical composition according to claim 1, wherein
the FP prostanoid receptor agonist is selected from the group which consists of prostaglandin, prostaglandin analogue and mixtures thereof.

3. The phosphate-free pharmaceutical composition according to claim 2, wherein
the prostaglandin is prostaglandin F₂ₐₗₚₕₐ or the pharmaceutically acceptable salts or esters thereof.

4. The phosphate-free pharmaceutical composition according to claim 2, wherein
the prostaglandin analogue is a prostaglandin F₂ₐₗₚₕₐ analogue or the pharmaceutically acceptable salts or esters thereof.

5. The phosphate-free pharmaceutical composition according to claim 4, wherein
the prostaglandin F₂ₐₗₚₕₐ analogue is selected from the group which consists of latanoprost, tafluprost, travopost, unoprostone and mixtures thereof and pharmaceutically acceptable salts and esters thereof.

6. The phosphate-free pharmaceutical composition according to claim 4,
wherein the prostaglandin F₂ₐₗₚₕₐ analogue is a 15-keto-prostaglandin F₂ₐₗₚₕₐ analogue.

7. The phosphate-free pharmaceutical composition according to claim 1,
wherein the prostamide receptor agonist is prostamide or a prostamide analogue.

8. The phosphate-free pharmaceutical composition according to claim 7,
wherein the prostamide analogue is a prostaglandin F₂ₐₗₚₕₐ amide, a 15-keto-prostaglandin F₂ₐₗₚₕₐ amide analogue or a pharmaceutically acceptable salt or a pharmaceutically acceptable ester thereof.

9. The phosphate-free pharmaceutical composition according to claim 8,
wherein the prostaglandin F₂ₐₗₚₕₐ amide has the chemical structural formula (VIII) 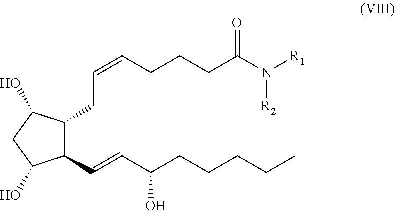
wherein R₁ and R₂ stand for, independently of each other, hydrogen, alkyl or hydroxyalkyl with 1 to 8 carbon atoms.

10. The phosphate-free pharmaceutical composition according to claim 9, wherein
R₁ stands for alkyl or hydroxyalkyl with 1 to 8 carbon atoms and R₂ stands for hydrogen.

11. The phosphate-free pharmaceutical composition according to claim 9,
wherein the prostaglandin F₂ₐₗₚₕₐ amide is present as 15-keto-prostaglandin F₂ₐₗₚₕₐ amide analogue.

12. The phosphate-free pharmaceutical composition according to claim 8,
wherein the prostaglandin F₂ₐₗₚₕₐ amide is prostaglandin F₂ₐₗₚₕₐ-1-ethanol amide or bimatoprost.

13. The phosphate-free pharmaceutical composition according to claim 1,
wherein the pharmaceutical composition comprises at least one ophthalmologically tolerable viscosity regulator.

14. The phosphate-free pharmaceutical composition according to claim 13, wherein
the ophthalmologically tolerable viscosity regulator is selected from the group which consists of chondroitin sulphate, polyacrylamide, polyacrylic acid, polyacrylic resins, polyethylene glycol, cellulose derivatives, polysaccharides, polyvinyl pyrrolidone, hyaluronic acid, hyaluronates, derivatives thereof and mixtures thereof.

15. The phosphate-free pharmaceutical composition according to claim 14,
wherein the hyaluronic acid, the hyaluronate and/or the derivatives thereof have a molecular weight which is in a range of approx. 50,000 to approx. 10,000,000 dalton.

16. The phosphate-free pharmaceutical composition according to claim 1,
wherein the pharmaceutical composition comprises phosphate-free pharmaceutical auxiliary substances which are selected from the group which consists of inorganic buffer substances, organic buffer substances, inorganic salts, organic salts, solvents, solubility aids, solubility promoters, salt formers, viscosity and consistency regulators, gelatinising agents, emulsifiers, solubilisers, wetting agents, expansion aids, antioxidants, preservatives, filling and carrier substances, osmolarity regulators and also mixtures thereof.

17. The phosphate-free pharmaceutical composition according to claim 1,
wherein the phosphate-free pharmaceutical composition is present in the form of a solution, of drops, a spray, a suspension, emulsion, a gel, an ointment, paste, a powder, powder, granulate or a tablet.

18-19. (canceled)

20. The phosphate-free pharmaceutical composition according to claim 1 which is supplied in a multidose container or multidose system.

21. A method of treating or preventing glaucoma in a patient comprising administering an effective amount of the phosphate-free pharmaceutical composition according to claim 1.
